(19) United States
(12) Patent Application Publication
(10) Pub. No.: US 2025/0279202 A1
(43) Pub. Date: Sep. 4, 2025

(54) ARTIFICIAL INTELLIGENCE BASED (AI-BASED) CANCER DETECTION SYSTEM AND METHOD FOR DETECTING CANCER IN PRECANCEROUS PHASES

(71) Applicant: Solstara, LLC, Wilmington, DE (US)

(72) Inventors: Faisal Nawaz, Brooklyn, NY (US); Logan Turley, Scottsdale, AZ (US)

(21) Appl. No.: 18/893,368

(22) Filed: Sep. 23, 2024

Related U.S. Application Data
(60) Provisional application No. 63/559,964, filed on Mar. 1, 2024.

(51) Int. Cl. G16H 50/20; A61B 5/00; A61B 5/055; G06F 40/279; G06T 7/00; G16B 20/00

(52) U.S. Cl. CPC G16H 50/20; A61B 5/055; A61B 5/7267; G06F 40/279; G06T 7/0014; G16B 20/00; G06T 2207/10088; G06T 2207/20081; G06T 2207/30096

(57) ABSTRACT

An artificial intelligence based (AI-based) cancer detection method and system for detecting cancer in precancerous phases is disclosed. The artificial intelligence based cancer detection method comprises: obtaining data from communication devices associated with first users, and databases; analyzing visual information associated with MRI scans of second users, indicating cancer symptoms by comparing medical data with historical medical records, using a computer vision model; analyzing textual information associated with blood test results and genetic information, of the second users, and the visual information, using an AI model; detecting a type of the cancer based on analysis of visual information associated with MRI scans of the second users, and textual information associated with the blood test results and genetic information, of the second users, using the AI model; and providing an output of the type of the cancer through user interfaces associated with the communication devices of the first users.

### EARLIEST PRIORITY DATE

This application claims priority from a Provisional patent application filed in the United States of America having Patent Application No. 63/559,964, filed on Mar. 1, 2024, and titled “ARTIFICIAL INTELLIGENCE-BASED SYSTEM AND METHOD FOR DETECTING CANCER IN PRECANCEROUS PHASES”.

### TECHNICAL FIELD

Embodiments of the present disclosure relate to automated medical analysis, and more particularly relate to an artificial intelligence based (AI-based) system and method for detecting cancer in precancerous phases.

### BACKGROUND

The field of cancer detection has witnessed significant advancements in recent years, primarily relying on imaging, biopsies, and blood tests. While these methods have been valuable in diagnosing cancer, the methods may have some limitations. Current practices often face challenges related to invasiveness, cost, and ability to detect cancer at its earliest stages, and potentially more treatable.

Imaging techniques, including at least one of: Magnetic resonance imaging (MRIs) and Computed Tomography (CT) scans, play a crucial role in cancer diagnosis. However, their sensitivity in detecting subtle abnormalities, especially during the precancerous phase, remains a challenge. Biopsies, a common diagnostic procedure, are invasive, potentially discomforting for patients, and may not always provide conclusive results. Blood tests, while offering non-invasive alternatives, may lack the precision needed for early detection.

Moreover, existing methods operate in silos, with individual specialists interpreting specific datasets independently. This compartmentalized approach leads to oversights and hinders the synthesis of a comprehensive understanding of the condition of a patient.

There are various technical problems with cancer detection methods in the prior art. In the existing technology, the cancer detection methods for the cancer detection are characterized by their invasiveness, high costs, and limited efficacy, particularly in the precancerous phases of the cancer. The existing method involves human review of various images, scans, and software visualizations, requiring manual assessment across diverse datasets.

Therefore, there is a need for an improved artificial intelligence based (AI-based) system and method for detecting cancer in precancerous phases, in order to address the aforementioned issues.

### SUMMARY

This summary is provided to introduce a selection of concepts, in a simple manner, which is further described in the detailed description of the disclosure. This summary is neither intended to identify key or essential inventive concepts of the subject matter nor to determine the scope of the disclosure.

In accordance with an embodiment of the present disclosure, an artificial intelligence based cancer detecting method for detecting cancer in precancerous phases, is disclosed. The artificial intelligence based cancer detecting method comprises obtaining, by one or more hardware processors, one or more data from at least one of: one or more communication devices associated with one or more first users and one or more databases. The one or more data comprise at least one of: one or more medical data and one or more historical medical records, associated with one or more second users. The one or more medical data associated with the one or more second users comprise at least one of: one or more magnetic resonance imaging (MRI) scans, one or more blood test results, and one or more genetic information, of the one or more second users.

The artificial intelligence based cancer detecting method further comprises analyzing, by the one or more hardware processors, one or more visual information associated with the magnetic resonance imaging (MRI) scans of the one or more second users, indicating cancer symptoms by comparing the one or more medical data with the one or more historical medical records, using a computer vision model. The artificial intelligence based cancer detecting method further comprises analyzing, by the one or more hardware processors, one or more textual information associated with at least one of: the one or more blood test results and the one or more genetic information, of the one or more second users, and the one or more visual information associated with the magnetic resonance imaging (MRI) scans being analyzed by the computer vision model, using an artificial intelligence (AI) model.

The artificial intelligence based cancer detecting method further comprises detecting, by the one or more hardware processors, a type of the cancer in the precancerous phases based on the analysis of the one or more visual information associated with the magnetic resonance imaging (MRI) scans of the one or more second users, and one or more textual information associated with at least one of: the one or more blood test results and the one or more genetic information, of the one or more second users, using the artificial intelligence (AI) model. The artificial intelligence based cancer detecting method further comprises providing, by the one or more hardware processors, an output of the type of the cancer in the precancerous phases in form of one or more reports through one or more user interfaces associated with the one or more communication devices of the one or more first users.

In an embodiment, detecting, by the one or more hardware processors, the type of the cancer in the precancerous phases comprises comparing the one or more data obtained from at least one of: the one or more communication devices associated with the one or more first users and the one or more databases, with one or more trained datasets obtained from the artificial intelligence (AI) model.

In another embodiment, analyzing the one or more visual information associated with the magnetic resonance imaging (MRI) scans of the one or more second users, using the computer vision model, comprises: (a) obtaining, by the one or more hardware processors, the one or more medical data comprising the one or more magnetic resonance imaging (MRI) scans of the one or more second users, from the one or more communication devices associated with the one or more first users and the one or more databases; (b) comparing, by the one or more hardware processors, the one or more medical data comprising the one or more magnetic resonance imaging (MRI) scans of the one or more second users, with the one or more historical medical records associated with the one or more second users, using the computer vision model; and (c) analyzing, by the one or more hardware processors, the one or more visual information associated with the magnetic resonance imaging (MRI) scans of the one or more second users, indicating the cancer symptoms, based on comparison of the one or more medical data comprising the one or more magnetic resonance imaging (MRI) scans of the one or more second users, with the one or more historical medical records associated with the one or more second users, using the computer vision model.

In yet another embodiment, the artificial intelligence based (AI-based) cancer detection method further comprises training, by the one or more hardware processors, the computer vision model based on one or more first training datasets associated with the one or more visual information of at least one of: the one or more medical data and the one or more historical medical records.

In yet another embodiment, the artificial intelligence based (AI-based) cancer detection method further comprises training, by the one or more hardware processors, the artificial intelligence (AI) model, by: (a) obtaining, by the one or more hardware processors, one or more second training datasets associated with at least one of: the one or more medical data and the one or more historical medical records, wherein the one or more second training datasets comprise the one or more first training datasets being processed by the computer vision model; (b) assigning, by the one or more hardware processors, one or more training weights to the one or more second training datasets based on priority of each data associated with at least one of: the one or more medical data and the one or more historical medical records; (c) fine-tuning, by the one or more hardware processors, the one or more training weights based on the one or more second training datasets indicating information associated with tumor lifecycle to determine whether a training subsystem configured with the artificial intelligence (AI) model analyzes one or more cancer patterns without bias from tumor progression stages; and (d) detecting, by the one or more hardware processors, the type of the cancer comprising at least one of: benign tumor lifecycle and malignant tumor lifecycle based on the fine-tuned one or more training weights assigned to the one or more second training datasets.

In yet another embodiment, the artificial intelligence based (AI-based) cancer detection method further comprises performing, by the one or more hardware processors, a reinforcement refinement process by assigning one or more tokens for each second training dataset to analyze each medical data, upon failure of detection of the malignant tumor lifecycle. In an embodiment, performing the reinforcement refinement process comprises retraining, by the one or more hardware processors, the artificial intelligence (AI) model with one or more corrected training weights integrating one or more feedback, to optimize an accuracy of the detection of the type of the cancer.

In yet another embodiment, the artificial intelligence based (AI-based) cancer detection method further comprises (a) obtaining, by the one or more hardware processors, the one or more data comprising the one or more textual information associated with at least one of: the one or more blood test results and the one or more genetic information, of the one or more second users; (b) analyzing, by the one or more hardware processors, at least one of: one or more terminologies, one or more concepts, and one or more contexts, associated with the type of the cancer from the one or more textual information associated with at least one of: the one or more blood test results and the one or more genetic information, of the one or more second users, using the artificial intelligence (AI) model, wherein the artificial intelligence (AI) model comprises one or more natural language processing (NLP) models; and (c) generating, by the one or more hardware processors, one or more insights associated with the type of the cancer from at least one of: the one or more medical data and the one or more historical medical records, based on the analysis of the at least one of: the one or more terminologies, the one or more concepts, and the one or more contexts, associated with the type of the cancer, using the artificial intelligence (AI) model.

In one aspect, an artificial intelligence based (AI-based) cancer detection system for detecting cancer in precancerous phases, is disclosed. The artificial intelligence based system comprises one or more hardware processors and a memory unit. The memory unit is operatively coupled to the one or more hardware processors. The memory unit comprises a plurality of subsystems in form of programmable instructions executable by the one or more hardware processors.

The plurality of subsystems comprises a data obtaining subsystem configured to one or more data from at least one of: one or more communication devices associated with one or more first users, and one or more databases. The one or more data comprise at least one of: one or more medical data and one or more historical medical records, associated with one or more second users. The one or more medical data associated with the one or more second users comprise at least one of: one or more magnetic resonance imaging (MRI) scans, one or more blood test results, and one or more genetic information, of the one or more second users.

The plurality of subsystems further comprises an information analyzing subsystem configured to analyze one or more visual information associated with the magnetic resonance imaging (MRI) scans of the one or more second users, indicating cancer symptoms by comparing the one or more medical data with the one or more historical medical records, using a computer vision model. The information analyzing subsystem is further configured to analyze one or more textual information associated with at least one of: the one or more blood test results and the one or more genetic information, of the one or more second users, and the one or more visual information associated with the magnetic resonance imaging (MRI) scans being analyzed by the computer vision model, using an artificial intelligence (AI) model.

The plurality of subsystems further comprises a cancer detection subsystem configured to detect a type of the cancer in the precancerous phases based on the analysis of the one or more visual information associated with the magnetic resonance imaging (MRI) scans of the one or more second users, and one or more textual information associated with at least one of: the one or more blood test results and the one or more genetic information, of the one or more second users, using the artificial intelligence (AI) model.

The plurality of subsystems further comprises an output subsystem configured to provide an output of the type of the cancer in the precancerous phases in form of one or more reports through one or more user interfaces associated with the one or more communication devices of the one or more first users.

In another aspect, a non-transitory computer-readable storage medium having instructions stored therein that, when executed by a hardware processor, causes the processor to perform method steps as described above.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF DRAWINGS

The disclosure will be described and explained with additional specificity and detail with the accompanying figures in which:

FIG. 1 illustrates an exemplary block diagram representation of a network architecture of an artificial intelligence based cancer detection system for detecting cancer in precancerous phases, in accordance with an embodiment of the present disclosure;

FIG. 2 illustrates a detailed view of the artificial intelligence based cancer detection system as shown in FIG. 1 for detecting the cancer in the precancerous phases, in accordance with an embodiment of the present disclosure;

FIG. 3 illustrates an exemplary flowchart depicting a training process of an artificial intelligence (AI) model (e.g., a Large Language Model (LLM)) for detecting the cancer in the precancerous phases, in accordance with an embodiment of the present disclosure;

FIGS. 4A-4G illustrate exemplary block diagrams depicting the training process of the artificial intelligence (AI) model (e.g., the Large Language Model (LLM)) for detecting the cancer in the precancerous phases, such as those in FIG. 3, in accordance with an embodiment of the present disclosure;

FIGS. 5A-5B illustrate exemplary visual representations depicting one or more user interfaces configured with one or more reports, in accordance with an embodiment of the present disclosure; and

FIG. 6 illustrates a flow diagram illustrating an artificial intelligence based cancer detection method for detecting the cancer in the precancerous phases, in accordance with an embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION OF THE DISCLOSURE

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure. It will be understood by those skilled in the art that the foregoing general description and the following detailed description are exemplary and explanatory of the disclosure and are not intended to be restrictive thereof.

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

The terms “comprise”, “comprising”, or any other variations thereof, are intended to cover a non-exclusive inclusion, such that one or more devices or sub-systems or elements or structures or components preceded by “comprises . . . a” does not, without more constraints, preclude the existence of other devices, sub-systems, additional sub-modules. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

A computer system (standalone, client or server computer system) configured by an application may constitute a “module” (or “subsystem”) that is configured and operated to perform certain operations. In one embodiment, the “module” or “subsystem” may be implemented mechanically or electronically, so a module include dedicated circuitry or logic that is permanently configured (within a special-purpose processor) to perform certain operations. In another embodiment, a “module” or “subsystem” may also comprise programmable logic or circuitry (as encompassed within a general-purpose processor or other programmable processor) that is temporarily configured by software to perform certain operations.

Accordingly, the term “module” or “subsystem” should be understood to encompass a tangible entity, be that an entity that is physically constructed permanently configured (hardwired) or temporarily configured (programmed) to operate in a certain manner and/or to perform certain operations described herein.

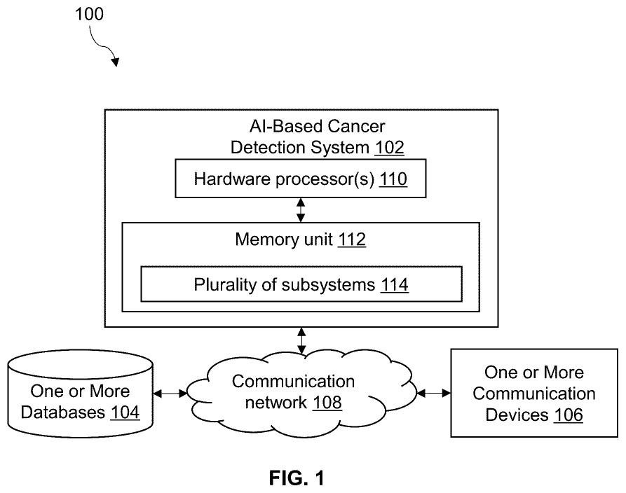

Referring now to the drawings, and more particularly to FIG. 1 through FIG. 6, where similar reference characters denote corresponding features consistently throughout the figures, there are shown preferred embodiments, and these embodiments are described in the context of the following exemplary system and/or method.

FIG. 1 illustrates an exemplary block diagram representation of a network architecture 100 of an artificial intelligence based cancer detection system 102 for detecting cancer in precancerous phases, in accordance with an embodiment of the present disclosure.

According to an exemplary embodiment of the present disclosure, FIG. 1 depicts the network architecture 100, which may include the artificial intelligence based cancer detection system 102, one or more databases 104, and one or more communication devices 106. The artificial intelligence based cancer detection system 102 may be communicatively coupled to the one or more databases 104, and the one or more communication devices 106 via a communication network 108. The communication network 108 may be a wired communication network and/or a wireless communication network. The one or more databases 104 may include, but not limited to, storing, and managing data related to one or more medical records. The one or more medical records may include, but not limited to, at least one of: scientific papers on the cancer, research papers on the cancer, doctor notes, and the like.

The artificial intelligence based cancer detection system 102 is initially configured to obtain one or more data from at least one of: the one or more communication devices 106 associated with one or more first users and one or more databases 104. In an embodiment, the one or more first users may include at least one of: one or more physicians, one or more doctors, one or more caretakers, one or more lab technicians, one or more healthcare professionals, and the like. In an embodiment, the one or more data may include at least one of: one or more medical data and one or more historical medical records (i.e., the one or more medical records), associated with one or more second users. In an embodiment, the one or more second users may include one or more patients. In an embodiment, the one or more medical data associated with the one or more second users may include at least one of: one or more magnetic resonance imaging (MRI) scans, one or more blood test results, one or more genetic information, of the one or more second users.

The artificial intelligence based cancer detection system 102 is further configured to analyze one or more visual information associated with the magnetic resonance imaging (MRI) scans of the one or more second users, indicating cancer symptoms by comparing the one or more medical data with the one or more historical medical records, using a computer vision model. The artificial intelligence based cancer detection system 102 is further configured to analyze one or more textual information associated with at least one of: the one or more blood test results and the one or more genetic information, of the one or more second users, and the one or more visual information associated with the magnetic resonance imaging (MRI) scans being analyzed by the computer vision model, using an artificial intelligence (AI) model.

The artificial intelligence based cancer detection system 102 is further configured to detect a type of the cancer in the precancerous phases based on the analysis of the one or more visual information associated with the magnetic resonance imaging (MRI) scans of the one or more second users, and one or more textual information associated with at least one of: the one or more blood test results and the one or more genetic information, of the one or more second users, using the artificial intelligence (AI) model. The artificial intelligence based cancer detection system 102 is further configured to provide an output of the type of the cancer in the precancerous phases in form of one or more reports through one or more user interfaces associated with the one or more communication devices 106 of the one or more first users.

The one or more databases 104 may be any kind of databases including, but not limited to, relational databases, non-relational databases, graph databases, document databases, dedicated databases, dynamic databases, monetized databases, scalable databases, cloud databases, distributed databases, any other databases, and a combination thereof. The one or more databases 104 are configured to support the functionality of the artificial intelligence based cancer detection system 102 and enables efficient data retrieval and storage for various aspects associated with the cancer detection.

In an exemplary embodiment, the one or more communication devices 106 may include, but not limited to, a mobile device, a smartphone, a Personal Digital Assistant (PDA), a tablet computer, a phablet computer, a wearable computing device, a laptop, a desktop, and the like.

This integrated network architecture 100 facilitates seamless communication and data exchange, enabling the artificial intelligence based cancer detection system 102 to operate cohesively for detecting the precancerous phases of the cancer using the one or more artificial intelligence models. The artificial intelligence based cancer detection system 102 may have capability to detect the precancerous phases of the cancer, thereby facilitating timely intervention and improving prognosis is underpinned by the effective collaboration among the artificial intelligence based cancer detection system 102, the one or more databases 104, and the one or more communication devices 106 within the communication network 108.

Further, the artificial intelligence based cancer detection system 102 may be implemented by way of a single device or a combination of multiple devices that may be operatively connected or networked together. The artificial intelligence based cancer detection system 102 may be implemented in hardware or a suitable combination of hardware and software. The artificial intelligence based cancer detection system 102 includes one or more hardware processors 110, and a memory unit 112. The memory unit 112 may include a plurality of subsystems 114. The artificial intelligence based cancer detection system 102 may be a hardware device including the one or more hardware processors 110 executing machine-readable program instructions for detecting the precancerous phases of the cancer. Execution of the machine-readable program instructions by the one or more hardware processors 110 may enable the artificial intelligence based cancer detection system 102 to dynamically recommend a course of action sequence for detecting the precancerous phases of the cancer.

The course of action sequences may involve various steps or decisions taken for data obtaining, Large Language Model (LLM) training, data scrutinizing, and report generation. The “hardware” may comprise a combination of discrete components, an integrated circuit, an application-specific integrated circuit, a field-programmable gate array, a digital signal processor, or other suitable hardware. The “software” may comprise one or more objects, agents, threads, lines of code, subroutines, separate software applications, two or more lines of code, or other suitable software structures operating in one or more software applications or on one or more processors.

The one or more hardware processors 110 may include, for example, microprocessors, microcomputers, microcontrollers, digital signal processors, central processing units, state machines, logic circuits, and/or any devices that manipulate data or signals based on operational instructions. Among other capabilities, the one or more hardware processors 110 may fetch and execute computer-readable instructions in the memory unit 112 operationally coupled with the artificial intelligence based cancer detection system 102 for performing tasks such as data processing, input/output processing, and/or any other functions. Any reference to a task in the present disclosure may refer to an operation being or that may be performed on data.

Though few components and the plurality of subsystems 114 are disclosed in FIG. 1, there may be additional components and subsystems which is not shown, such as, but not limited to, ports, routers, repeaters, firewall devices, network devices, databases, network attached storage devices, servers, assets, machinery, instruments, facility equipment, emergency management devices, image capturing devices, any other devices, and combination thereof. A person skilled in the art should not be limiting the components/subsystems shown in FIG. 1.

Those of ordinary skilled in the art will appreciate that the hardware depicted in FIG. 1 may vary for particular implementations. For example, other peripheral devices such as an optical disk drive and the like, local area network (LAN), wide area network (WAN), wireless (e.g., wireless-fidelity (Wi-Fi)) adapter, graphics adapter, disk controller, input/output (I/O) adapter also may be used in addition or place of the hardware depicted. The depicted example is provided for explanation only and is not meant to imply architectural limitations concerning the present disclosure.

Those skilled in the art will recognize that, for simplicity and clarity, the full structure and operation of all data processing systems suitable for use with the present disclosure are not being depicted or described herein. Instead, only so much of the artificial intelligence based cancer detection system 102 as is unique to the present disclosure or necessary for an understanding of the present disclosure is depicted and described. The remainder of the construction and operation of the artificial intelligence based cancer detection system 102 may conform to any of the various current implementations and practices that were known in the art.

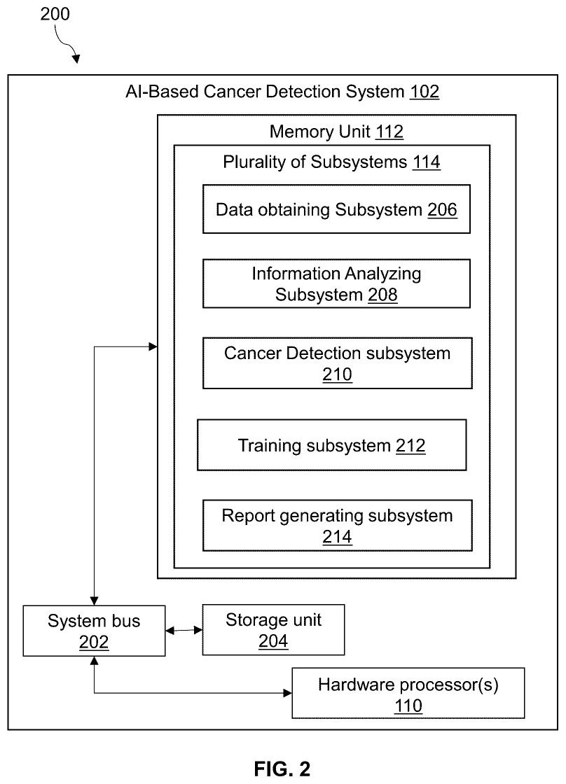

FIG. 2 illustrates a detailed view 200 of the artificial intelligence based cancer detection system 102 for detecting the cancer in the precancerous phases, such as those shown in FIG. 1, in accordance with an embodiment of the present disclosure.

The artificial intelligence based cancer detection system 102 includes the one or more hardware processors 110, the memory unit 112, and a storage unit 204. The one or more hardware processors 110, the memory unit 112, and the storage unit 204 are communicatively coupled through a system bus 202 or any similar mechanism. The memory unit 112 is operatively coupled to the one or more hardware processors 110. The memory unit 112 includes the plurality of subsystems 114 in the form of programmable instructions executable by the one or more hardware processors 110.

In an exemplary embodiment, the plurality of subsystems 114 comprises a data obtaining subsystem 206, an information analyzing subsystem 208, a cancer detection subsystem 210, a training subsystem 212, and a report generating subsystem 214.

The one or more hardware processors 110, as used herein, means any type of computational circuit, such as, but not limited to, a microprocessor unit, microcontroller, complex instruction set computing microprocessor unit, reduced instruction set computing microprocessor unit, very long instruction word microprocessor unit, explicitly parallel instruction computing microprocessor unit, graphics processing unit, digital signal processing unit, or any other type of processing circuit. The one or more hardware processors 110 may also include embedded controllers, such as generic or programmable logic devices or arrays, application-specific integrated circuits, single-chip computers, and the like.

The memory unit 112 may be a non-transitory volatile memory and a non-volatile memory. The memory unit 112 may be coupled to communicate with the one or more hardware processors 110, such as being a computer-readable storage medium. The one or more hardware processors 110 may execute machine-readable instructions and/or source code stored in the memory unit 112. A variety of machine-readable instructions may be stored in and accessed from the memory unit 112. The memory unit 112 may include any suitable elements for storing data and machine-readable instructions, such as read-only memory, random access memory, erasable programmable read-only memory, electrically erasable programmable read-only memory, a hard drive, a removable media drive for handling compact disks, digital video disks, diskettes, magnetic tape cartridges, memory cards, and the like. In the present embodiment, the memory unit 112 includes the plurality of subsystems 114 stored in the form of machine-readable instructions on any of the above-mentioned storage media and may be in communication with and executed by the one or more hardware processors 110.

The storage unit 204 may be a cloud storage or the one or more databases 104 such as those shown in FIG. 1. The storage unit 204 may store, but not limited to, recommending a course of action sequences, applications, application links, application name, application description, application meta-data, application identifier, display names of the one or more applications, short textual description, a universal resource locator (URL) of the one or more applications, and a list of parameters corresponding to an application context, generated recommending course of action sequences, one or more clickable elements, completion status of initiated user action through recommended course of action sequences, feedback loops, feedback from one or more users, query parameters, additional query parameters, deep integration parameters, up-sell/x-sell product links, tracked user click-through rates, any other data, and combinations thereof. The storage unit 204 may be any kind of database such as, but not limited to, relational databases, dedicated databases, dynamic databases, monetized databases, scalable databases, cloud databases, distributed databases, any other databases, and a combination thereof.

In an exemplary embodiment, the user interface subsystem 206 is configured with the user interface associated with the one or more communication devices 106. The user interface is configured to allow the one or more users to interact with the system 102. The user interface subsystem 206 is configured to allow the one or more users to input medical data. The medical data may include, but not limited to, at least one of: Magnetic Resonance Imaging (MRI) scans, blood test results, genetic information, and the like. The one or more users may upload the medical data through form filling, file uploading, and the like provided by the user interface. The user interface may include, but not limited to, a chatbox, and the like.

The plurality of subsystems 114 includes the data obtaining subsystem 206 that is communicatively connected to the one or more hardware processors 110. The data obtaining subsystem 206 is configured to obtain the one or more data from at least one of: the one or more communication devices 106 associated with the one or more first users and the one or more databases 104. The one or more data may include at least one of: the one or more medical data and the one or more medical records (i.e., the one or more historical medical records), associated with the one or more second users. In an embodiment, the one or more medical data associated with the one or more second users may include at least one of: the one or more magnetic resonance imaging (MRI) scans, the one or more blood test results, the one or more genetic information, of the one or more second users. The data obtaining subsystem 206 serves as a foundation for the system 102, acquiring the medical data and the medical records necessary for further analysis.

In an embodiment, the data obtaining subsystem 206 is configured to obtain the one or more data through one or more user interfaces associated with the one or more communication devices 106. The one or more user interfaces associated with the one or more communication devices 106 are configured to adapt the one or more first users to interact with the artificial intelligence based cancer detection system 102. The one or more user interfaces are further configured to adapt the one or more first users to input the one or more medical data. The one or more second users may upload the one or more medical data through form filling, file uploading, and the like provided by the one or more user interfaces. The one or more user interfaces may include, but not limited to, a chatbox, and the like.

The plurality of subsystems 114 further includes the information analyzing subsystem 208 that is communicatively connected to the one or more hardware processors 110. The information analyzing subsystem 208 is configured to analyze the one or more visual information associated with the magnetic resonance imaging (MRI) scans of the one or more second users, indicating cancer symptoms by comparing the one or more medical data with the one or more historical medical records, using the computer vision model.

In an embodiment, for analyzing the one or more visual information associated with the magnetic resonance imaging (MRI) scans of the one or more second users, using the computer vision model, the information analyzing subsystem 208 is configured to obtain the one or more medical data including the one or more magnetic resonance imaging (MRI) scans of the one or more second users, from the one or more communication devices 106 associated with the one or more first users and the one or more databases 104. The information analyzing subsystem 208 is further configured to compare the one or more medical data including the one or more magnetic resonance imaging (MRI) scans of the one or more second users, with the one or more historical medical records associated with the one or more second users, using the computer vision model. The information analyzing subsystem 208 is further configured to analyze the one or more visual information associated with the magnetic resonance imaging (MRI) scans of the one or more second users, indicating the cancer symptoms, based on comparison of the one or more medical data including the one or more magnetic resonance imaging (MRI) scans of the one or more second users, with the one or more historical medical records associated with the one or more second users, using the computer vision model.

In an embodiment, the information analyzing subsystem 208 is configured to analyze the one or more textual information associated with at least one of: the one or more blood test results and the one or more genetic information, of the one or more second users, and the one or more visual information associated with the magnetic resonance imaging (MRI) scans being analyzed by the computer vision model, using an artificial intelligence (AI) model.

The plurality of subsystems 114 further includes the cancer detection subsystem 210 that is communicatively connected to the one or more hardware processors 110. The cancer detection subsystem 210 is configured to detect the type of the cancer in the precancerous phases based on the analysis of the one or more visual information associated with the magnetic resonance imaging (MRI) scans of the one or more second users, and one or more textual information associated with at least one of: the one or more blood test results and the one or more genetic information, of the one or more second users, using the artificial intelligence (AI) model.

In an embodiment, for detecting the type of the cancer in the precancerous phases, the cancer detection subsystem 210 is configured to compare the one or more data obtained from at least one of: the one or more communication devices 106 associated with the one or more first users and the one or more databases 104, with one or more trained datasets obtained from the artificial intelligence (AI) model. The comparison of the one or more medical data and the one or more trained datasets to diligently assess information about the potential precancerous phases. The cancer detection subsystem 210 is configured to scrutinize the one or more medical data, with precision and accuracy, and scrutinized data is generated. The cancer detection subsystem 210 is further configured to enhance diagnostic capabilities, swiftly detecting subtle abnormalities indicative of the cancer based on the one or more trained datasets. The artificial intelligence based (AI-based) cancer detection system 102 excels in providing accurate diagnoses and the prognoses, empowering healthcare professionals with comprehensive and actionable insights to enhance the cancer diagnosis, treatment planning, and patient care.

The plurality of subsystems 114 further includes the training subsystem 212 that is communicatively connected to the one or more hardware processors 110. The training subsystem 212 is configured to train the computer vision model based on one or more first training datasets associated with the one or more visual information of at least one of: the one or more medical data and the one or more historical medical records.

The training subsystem 212 is further configured to train the artificial intelligence (AI) model. In an embodiment, the artificial intelligence (AI) model may be a Large Language Model (LLM) and a Natural Language Processing (NLP) model. In an embodiment, the training of the artificial intelligence (AI) model undergoes a meticulous training process, wherein the extensive one or more medical records serve as the foundation. The one or more medical records, meticulously curated, are strategically employed to refine the focus of the artificial intelligence based (Al-based) cancer detection system 102 specifically towards addressing challenges related to cancer detection. The training subsystem 212 relies on the one or more medical data from at least one of: the one or more first users and the one or more second users continuously, ensuring that the LLM is trained on the most up-to-date and relevant information. Furthermore, the artificial intelligence (AI) model (e.g., the LLM model) is uniquely designed to process and analyze the one or more textual information derived from the one or more medical data and the one or more medical records.

For training the artificial intelligence (AI) model, the training subsystem 212 is configured to obtain one or more second training datasets associated with at least one of: the one or more medical data and the one or more historical medical records. In an embodiment, the one or more second training datasets may include the one or more first training datasets being processed by the computer vision model. The training subsystem 212 is further configured to assign one or more training weights to the one or more second training datasets based on priority of each data associated with at least one of: the one or more medical data and the one or more historical medical records.

The training subsystem 212 is further configured to fine-tune the one or more training weights based on the one or more second training datasets indicating information associated with tumor lifecycle to determine whether the training subsystem 212 configured with the artificial intelligence (AI) model analyzes one or more cancer patterns without bias from tumor progression stages. The training subsystem 212 is further configured to detect the type of the cancer comprising at least one of: benign tumor lifecycle and malignant tumor lifecycle based on the fine-tuned one or more training weights assigned to the one or more second training datasets.

In an embodiment, the training subsystem 212 is further configured to perform a reinforcement refinement process by assigning one or more tokens for each second training dataset to analyze each medical data, upon failure of detection of the malignant tumor lifecycle. In an embodiment, for performing the reinforcement refinement process, the training subsystem 212 is configured to retrain the artificial intelligence (AI) model with one or more corrected training weights integrating one or more feedback (e.g., one or more feedback from one or more users), to optimize an accuracy of the detection of the type of the cancer. The training subsystem 212 is then configured to refine the one or more trained datasets.

In an embodiment, the training subsystem 212 is configured to obtain the one or more data including the one or more textual information associated with at least one of: the one or more blood test results and the one or more genetic information, of the one or more second users. The training subsystem 212 is further configured to analyze at least one of: one or more terminologies, one or more concepts, and one or more contexts, associated with the type of the cancer from the one or more textual information associated with at least one of: the one or more blood test results and the one or more genetic information, of the one or more second users, using the artificial intelligence (AI) model. In an embodiment, the artificial intelligence (AI) model may include one or more natural language processing (NLP) models. The training subsystem 212 is further configured to generate one or more insights associated with the type of the cancer from at least one of: the one or more medical data and the one or more historical medical records, based on the analysis of the at least one of: the one or more terminologies, the one or more concepts, and the one or more contexts, associated with the type of the cancer, using the artificial intelligence (AI) model.

The artificial intelligence (AI) model is configured to scrutinize the one or more textual information of the one or more medical data including one or more blood results, the one or more genetic information, and the like. The training subsystem 212 is configured to ensure the artificial intelligence (AI) model is finely tuned to interpret the diverse medical data, enabling the artificial intelligence (AI) model to provide the accurate and timely diagnoses of the one or more textual information, thereby enhancing care of the one or more users and clinical decision-making.

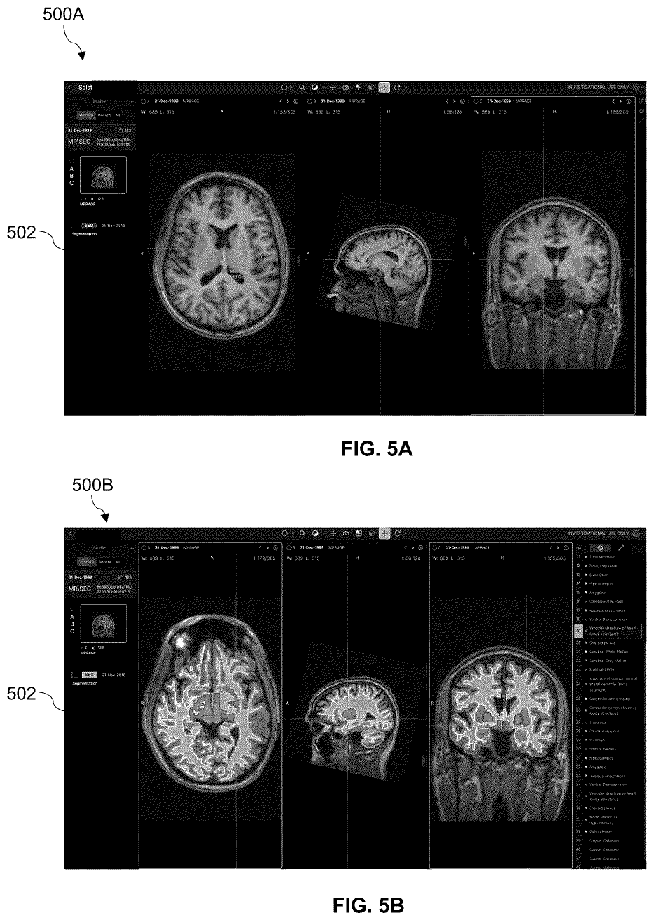

The plurality of subsystems 114 further includes the report generating subsystem 214 that is communicatively connected to the one or more hardware processors 110. The report generating subsystem 214 is configured to provide the output of the type of the cancer in the precancerous phases in form of the one or more reports through the one or more user interfaces associated with the one or more communication devices 106 of the one or more first users. An application programming interface (API) serves as the one or more interfaces for communication and data exchange between the one or more user interfaces and at least one of a: web application and mobile application. In other words, the report generating subsystem 214 is configured to provide the scrutinized data in the form of the report (as shown in FIG. 5A and FIG. 5B) to the one or more first users. The one or more user interfaces are configured to provide a centralized platform where the one or more first users is able to view, interact, and manage the one or more reports efficiently.

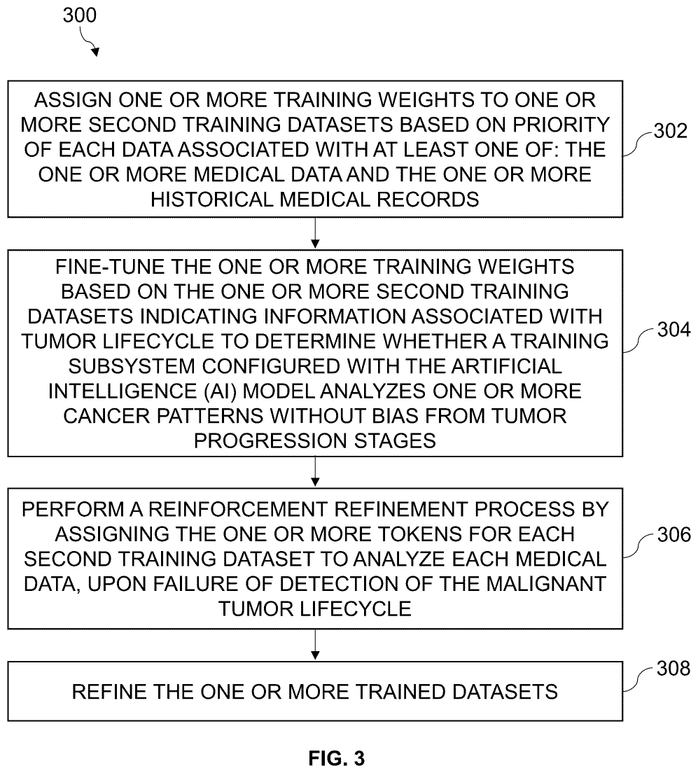

FIG. 3 illustrates an exemplary flowchart 300 depicting a training process of an artificial intelligence (AI) model (e.g., a Large Language Model (LLM)) for detecting the cancer in the precancerous phases, in accordance with an embodiment of the present disclosure. At step 302, the one or more training weights are assigned to the one or more second training datasets based on priority of each data associated with at least one of: the one or more medical data and the one or more historical medical records.

At step 304, the one or more training weights are fine-tuned based on the one or more second training datasets indicating the information associated with the tumor lifecycle to determine whether the training subsystem 212 configured with the artificial intelligence (AI) model analyzes the one or more cancer patterns without the bias from the tumor progression stages.

At step 306, the reinforcement refinement process is performed by assigning the one or more tokens for each second training dataset to analyze each medical data, upon failure of detection of the malignant tumor lifecycle. In an embodiment, for performing the reinforcement refinement process, the artificial intelligence (AI) model is retrained with the one or more corrected training weights integrating the one or more feedback (e.g., one or more feedback from one or more users), to optimize an accuracy of the detection of the type of the cancer. At step 308, the one or more trained datasets are refined.

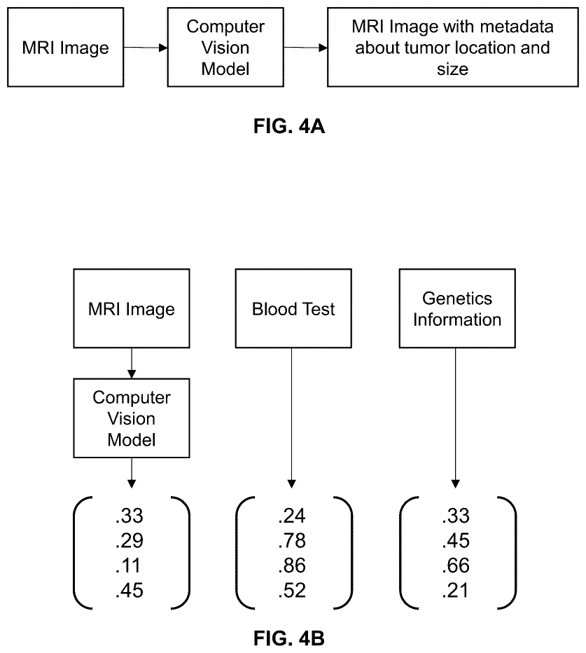

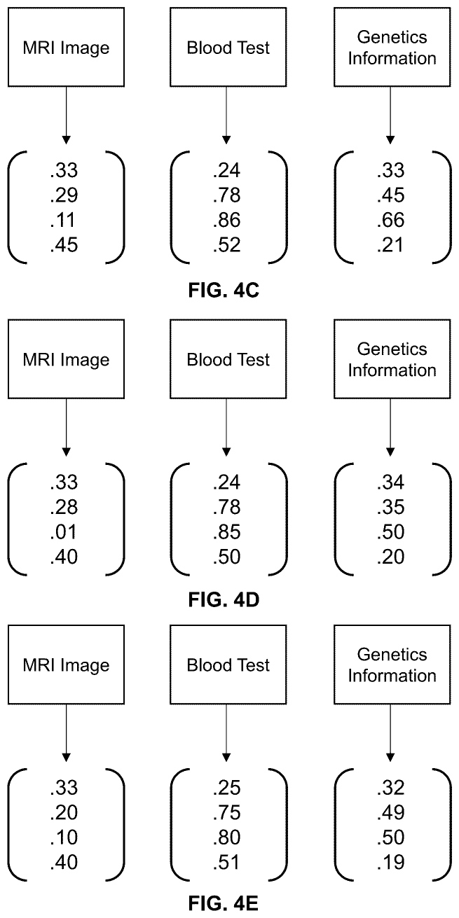

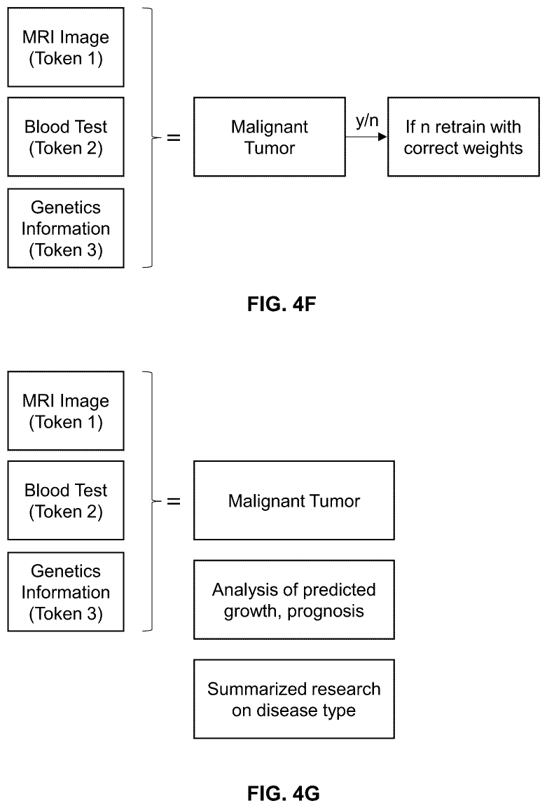

FIGS. 4A-4G illustrate exemplary block diagrams depicting the training process of the artificial intelligence (AI) model (e.g., the Large Language Model (LLM)) for detecting the cancer in the precancerous phases, such as those in FIG. 3, in accordance with an embodiment of the present disclosure. The details of the training process of the artificial intelligence (AI) model, shown in FIGS. 4A-4G, have been elaborated in above said FIG. 2 and FIG. 3.

FIGS. 5A-5B illustrate exemplary visual representations (500A, 500B) depicting one or more user interfaces 502 configured with one or more reports, in accordance with an embodiment of the present disclosure. In an exemplary embodiment, the one or more user interfaces 502 depicted in FIGS. 5A-5B, are configured with the one or more reports of the MRI scan. The one or more user interfaces 502 are designed to present the one or more reports in a clear and accessible manner, allowing the one or more first users to easily interpret and analyze the one or more reports of the MRI scan generated by the report generating subsystem 214. By integrating a MRI scan report into the one or more user interfaces 502, the one or more second users (e.g., the one or more healthcare professionals) conveniently access and review the findings of the one or more reports, thereby facilitating informed decision-making regarding medical treatment and further diagnostic procedures.

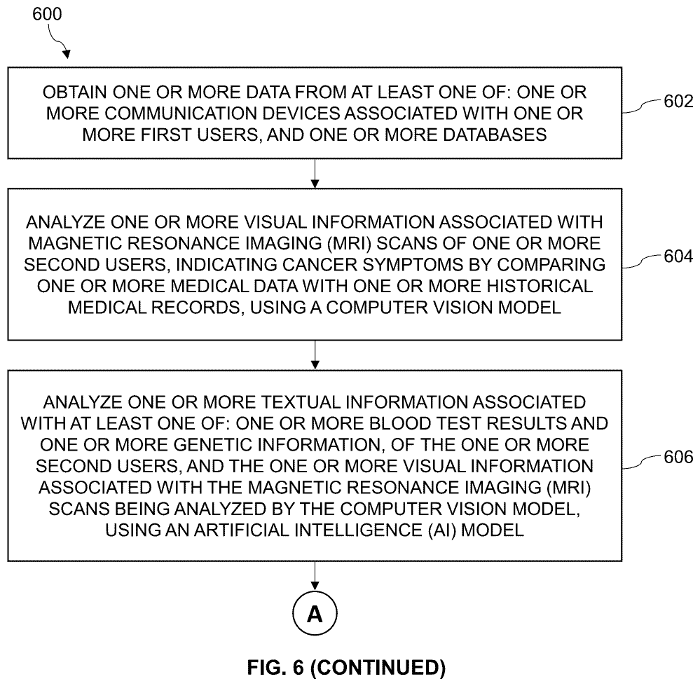

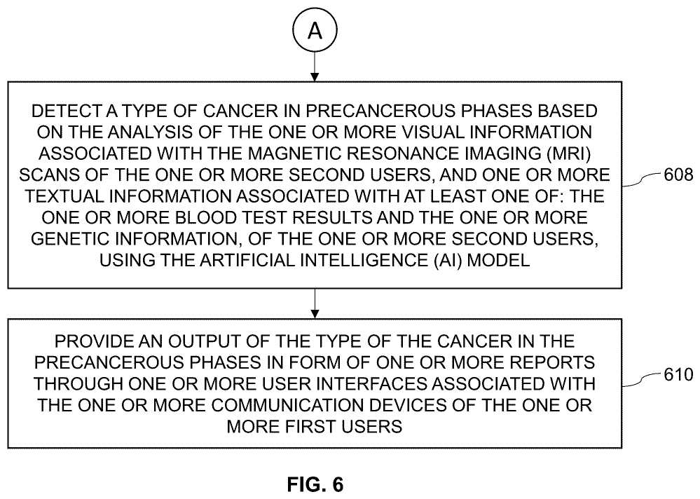

FIG. 6 illustrates a flow diagram illustrating an artificial intelligence based cancer detection method 600 for detecting the cancer in the precancerous phases, in accordance with an embodiment of the present disclosure.

At step 602, the one or more data are obtained from at least one of: the one or more communication devices 106 associated with the one or more first users, and the one or more databases 104. In an embodiment, the one or more data may include at least one of: the one or more medical data and the one or more historical medical records, associated with the one or more second users. In an embodiment, the one or more medical data associated with the one or more second users may include at least one of: the one or more magnetic resonance imaging (MRI) scans, the one or more blood test results, and the one or more genetic information, of the one or more second users.

At step 604, the one or more visual information associated with the magnetic resonance imaging (MRI) scans of the one or more second users, indicating cancer symptoms, are analyzed by comparing the one or more medical data with the one or more historical medical records, using the computer vision model.

At step 606, the one or more textual information associated with at least one of: the one or more blood test results and the one or more genetic information, of the one or more second users, and the one or more visual information associated with the magnetic resonance imaging (MRI) scans being analyzed by the computer vision model, are analyzed using the artificial intelligence (AI) model.

At step 608, the type of the cancer in the precancerous phases are detected based on the analysis of the one or more visual information associated with the magnetic resonance imaging (MRI) scans of the one or more second users, and one or more textual information associated with at least one of: the one or more blood test results and the one or more genetic information, of the one or more second users, using the artificial intelligence (AI) model.

At step 610, the output of the type of the cancer in the precancerous phases in form of one or more reports, is provided through the one or more user interfaces 502 associated with the one or more communication devices 106 of the one or more first users.

The present invention has following advantages. In accordance with the present disclosure, the artificial intelligence based (AI-based) cancer detection system 102 for detecting the early stages of the cancer is disclosed. The artificial intelligence based (AI-based) cancer detection system 102 is configured to deliver the precise prognostic insights spanning across an entire body of the one or more second users, ensuring comprehensive and tailored healthcare planning. The artificial intelligence based (AI-based) cancer detection system 102 is adept at predicting a current tumor presence as well as forecasting the likelihood of the future tumor development. The one or more first users may navigate complex transitions between primary care, oncology, radiology, and surgery, managing information independently. The intelligence based (AI-based) cancer detection system 102 is configured to consolidate the diverse medical data, empowering the one or more first users with a unified platform for streamlined care management. The intelligence based (AI-based) cancer detection system 102 is non-invasive and provides a cost-effective solution.

By analyzing the MRI scan, the intelligence based (AI-based) cancer detection system 102 may assist in hypothesis validation, enhancing research integrity. The intelligence based (AI-based) cancer detection system 102 is configured to aid in the curation of treatment plans by leveraging the one or more insights from the latest scientific papers. The intelligence based (AI-based) cancer detection system 102 is configured to facilitate the implementation of lifestyle modifications aimed at the cancer treatment. The intelligence based (AI-based) cancer detection system 102 is further configured to aid in analyzing radiology images, enhancing diagnostic accuracy and efficiency.

The intelligence based (AI-based) cancer detection system 102 is further configured to assist in identifying potential drug targets that may be suitable for therapy. The intelligence based (AI-based) cancer detection system 102 is configured to deliver the one or more first users with tailored education materials, ensuring information is personalized to their needs. The intelligence based (Al-based) cancer detection system 102 is further configured to assist in the interpretation and evaluation of clinical trial results. Moreover, the intelligence based (AI-based) cancer detection system 102 is further configured to facilitate rapid and accurate retrieval of the medical records, enhancing workflow efficiency.

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

The embodiments herein can comprise hardware and software elements. The embodiments that are implemented in software include but are not limited to, firmware, resident software, microcode, etc. The functions performed by various modules described herein may be implemented in other modules or combinations of other modules. For the purposes of this description, a computer-usable or computer-readable medium can be any apparatus that can comprise, store, communicate, propagate, or transport the program for use by or in connection with the instruction execution system, apparatus, or device.

The medium can be an electronic, magnetic, optical, electromagnetic, infrared, or semiconductor system (or apparatus or device) or a propagation medium. Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W) and DVD.

Input/output (I/O) devices (including but not limited to keyboards, displays, pointing devices, etc.) can be coupled to the artificial intelligence based (AI-based) cancer detection system 102 either directly or through intervening I/O controllers. Network adapters may also be coupled to the artificial intelligence based (AI-based) cancer detection system 102 to enable the data processing system to become coupled to other data processing systems or remote printers or storage devices through intervening private or public networks. Modems, cable modem and Ethernet cards are just a few of the currently available types of network adapters.

A representative hardware environment for practicing the embodiments may include a hardware configuration of an information handling/artificial intelligence based (AI-based) cancer detection system 102 in accordance with the embodiments herein. The artificial intelligence based (AI-based) cancer detection system 102 herein comprises at least one processor or central processing unit (CPU). The CPUs are interconnected via the system bus 202 to various devices including at least one of: a random-access memory (RAM), read-only memory (ROM), and an input/output (I/O) adapter. The I/O adapter can connect to peripheral devices, including at least one of: disk units and tape drives, or other program storage devices that are readable by the artificial intelligence based (Al-based) cancer detection system 102. The artificial intelligence based (AI-based) cancer detection system 102 can read the inventive instructions on the program storage devices and follow these instructions to execute the methodology of the embodiments herein.

The artificial intelligence based (AI-based) cancer detection system 102 further includes a user interface adapter that connects a keyboard, mouse, speaker, microphone, and/or other user interface devices including a touch screen device (not shown) to the bus to gather user input. Additionally, a communication adapter connects the bus to a data processing network, and a display adapter connects the bus to a display device which may be embodied as an output device including at least one of: a monitor, printer, or transmitter, for example.

A description of an embodiment with several components in communication with each other does not imply that all such components are required. On the contrary, a variety of optional components are described to illustrate the wide variety of possible embodiments of the invention. When a single device or article is described herein, it will be apparent that more than one device/article (whether or not they cooperate) may be used in place of a single device/article. Similarly, where more than one device or article is described herein (whether or not they cooperate), it will be apparent that a single device/article may be used in place of the more than one device or article, or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments. Also, the words “comprising,” “having,” “containing,” and “including,” and other similar forms are intended to be equivalent in meaning and be open-ended in that an item or items following any one of these words is not meant to be an exhaustive listing of such item or items or meant to be limited to only the listed item or items. It must also be noted that as used herein and in the appended claims, the singular forms “a,” “an,” and “the” include plural references unless the context clearly dictates otherwise.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the embodiments of the present invention are intended to be illustrative, but not limiting, of the scope of the invention, which is set forth in the following claims.

What is claimed is:

1. An artificial intelligence based (AI-based) cancer detection method for detecting cancer in precancerous phases, the artificial intelligence based (AI-based) cancer detection method comprising:
obtaining, by one or more hardware processors, one or more data from at least one of: one or more communication devices associated with one or more first users, and one or more databases, wherein the one or more data comprise at least one of: one or more medical data and one or more historical medical records, associated with one or more second users, and wherein the one or more medical data associated with the one or more second users comprise at least one of: one or more magnetic resonance imaging (MRI) scans, one or more blood test results, and one or more genetic information, of the one or more second users;
analyzing, by the one or more hardware processors, one or more visual information associated with the magnetic resonance imaging (MRI) scans of the one or more second users, indicating cancer symptoms by comparing the one or more medical data with the one or more historical medical records, using a computer vision model;
analyzing, by the one or more hardware processors, one or more textual information associated with at least one of: the one or more blood test results and the one or more genetic information, of the one or more second users, and the one or more visual information associated with the magnetic resonance imaging (MRI) scans being analyzed by the computer vision model, using an artificial intelligence (AI) model;
detecting, by the one or more hardware processors, a type of the cancer in the precancerous phases based on the analysis of the one or more visual information associated with the magnetic resonance imaging (MRI) scans of the one or more second users, and one or more textual information associated with at least one of: the one or more blood test results and the one or more genetic information, of the one or more second users, using the artificial intelligence (AI) model; and
providing, by the one or more hardware processors, an output of the type of the cancer in the precancerous phases in form of one or more reports through one or more user interfaces associated with the one or more communication devices of the one or more first users.

2. The artificial intelligence based (AI-based) cancer detection method of claim 1, wherein detecting, by the one or more hardware processors, the type of the cancer in the precancerous phases comprises comparing the one or more data obtained from at least one of: the one or more communication devices associated with the one or more first users and the one or more databases, with one or more trained datasets obtained from the artificial intelligence (AI) model.

3. The artificial intelligence based (AI-based) cancer detection method of claim 1, wherein analyzing the one or more visual information associated with the magnetic resonance imaging (MRI) scans of the one or more second users, using the computer vision model, comprises:
obtaining, by the one or more hardware processors, the one or more medical data comprising the one or more magnetic resonance imaging (MRI) scans of the one or more second users, from the one or more communication devices associated with the one or more first users and the one or more databases;
comparing, by the one or more hardware processors, the one or more medical data comprising the one or more magnetic resonance imaging (MRI) scans of the one or more second users, with the one or more historical medical records associated with the one or more second users, using the computer vision model; and
analyzing, by the one or more hardware processors, the one or more visual information associated with the magnetic resonance imaging (MRI) scans of the one or more second users, indicating the cancer symptoms, based on comparison of the one or more medical data comprising the one or more magnetic resonance imaging (MRI) scans of the one or more second users, with the one or more historical medical records associated with the one or more second users, using the computer vision model.

4. The artificial intelligence based (AI-based) cancer detection method of claim 3, further comprising training, by the one or more hardware processors, the computer vision model based on one or more first training datasets associated with the one or more visual information of at least one of: the one or more medical data and the one or more historical medical records.

5. The artificial intelligence based (AI-based) cancer detection method of claim 1, further comprising training, by the one or more hardware processors, the artificial intelligence (AI) model, by:
obtaining, by the one or more hardware processors, one or more second training datasets associated with at least one of: the one or more medical data and the one or more historical medical records, wherein the one or more second training datasets comprise the one or more first training datasets being processed by the computer vision model;
assigning, by the one or more hardware processors, one or more training weights to the one or more second training datasets based on priority of each data associated with at least one of: the one or more medical data and the one or more historical medical records;
fine-tuning, by the one or more hardware processors, the one or more training weights based on the one or more second training datasets indicating information associated with tumor lifecycle to determine whether a training subsystem configured with the artificial intelligence (AI) model analyzes one or more cancer patterns without bias from tumor progression stages; and
detecting, by the one or more hardware processors, the type of the cancer comprising at least one of: benign tumor lifecycle and malignant tumor lifecycle based on the fine-tuned one or more training weights assigned to the one or more second training datasets.

6. The artificial intelligence based (AI-based) cancer detection method of claim 5, further comprising performing, by the one or more hardware processors, a reinforcement refinement process by assigning one or more tokens for each second training dataset to analyze each medical data, upon failure of detection of the malignant tumor lifecycle,
wherein performing the reinforcement refinement process comprises retraining, by the one or more hardware processors, the artificial intelligence (AI) model with one or more corrected training weights integrating one or more feedback, to optimize an accuracy of the detection of the type of the cancer.

7. The artificial intelligence based (AI-based) cancer detection method of claim 1, further comprising:
obtaining, by the one or more hardware processors, the one or more data comprising the one or more textual information associated with at least one of: the one or more blood test results and the one or more genetic information, of the one or more second users;
analyzing, by the one or more hardware processors, at least one of: one or more terminologies, one or more concepts, and one or more contexts, associated with the type of the cancer from the one or more textual information associated with at least one of: the one or more blood test results and the one or more genetic information, of the one or more second users, using the artificial intelligence (AI) model, wherein the artificial intelligence (AI) model comprises one or more natural language processing (NLP) models; and
generating, by the one or more hardware processors, one or more insights associated with the type of the cancer from at least one of: the one or more medical data and the one or more historical medical records, based on the analysis of the at least one of: the one or more terminologies, the one or more concepts, and the one or more contexts, associated with the type of the cancer, using the artificial intelligence (AI) model.

8. An artificial intelligence based (AI-based) cancer detection system for detecting cancer in precancerous phases, the artificial intelligence based (AI-based) cancer detection system comprising:
one or more hardware processors;
a memory coupled to the one or more hardware processors, wherein the memory comprises a plurality of subsystems in form of programmable instructions executable by the one or more hardware processors, and wherein the plurality of subsystems comprises:
a data obtaining subsystem configured to one or more data from at least one of: one or more communication devices associated with one or more first users, and one or more databases, wherein the one or more data comprise at least one of: one or more medical data and one or more historical medical records, associated with one or more second users, and wherein the one or more medical data associated with the one or more second users comprise at least one of: one or more magnetic resonance imaging (MRI) scans, one or more blood test results, and one or more genetic information, of the one or more second users;
an information analyzing subsystem configured to:
analyze one or more visual information associated with the magnetic resonance imaging (MRI) scans of the one or more second users, indicating cancer symptoms by comparing the one or more medical data with the one or more historical medical records, using a computer vision model; and
analyze one or more textual information associated with at least one of: the one or more blood test results and the one or more genetic information, of the one or more second users, and the one or more visual information associated with the magnetic resonance imaging (MRI) scans being analyzed by the computer vision model, using an artificial intelligence (AI) model;
a cancer detection subsystem configured to detect a type of the cancer in the precancerous phases based on the analysis of the one or more visual information associated with the magnetic resonance imaging (MRI) scans of the one or more second users, and one or more textual information associated with at least one of: the one or more blood test results and the one or more genetic information, of the one or more second users, using the artificial intelligence (AI) model; and
an output subsystem configured to provide an output of the type of the cancer in the precancerous phases in form of one or more reports through one or more user interfaces associated with the one or more communication devices of the one or more first users.

9. The artificial intelligence based (AI-based) cancer detection system of claim 8, wherein in detecting the type of the cancer in the precancerous phases, the cancer detection subsystem is further configured to compare the one or more data obtained from at least one of: the one or more communication devices associated with the one or more first users and the one or more databases, with one or more trained datasets obtained from the artificial intelligence (AI) model.

10. The artificial intelligence based (AI-based) cancer detection system of claim 8, wherein in analyzing the one or more visual information associated with the magnetic resonance imaging (MRI) scans of the one or more second users, using the computer vision model, the information analyzing subsystem is further configured to:
obtain the one or more medical data comprising the one or more magnetic resonance imaging (MRI) scans of the one or more second users, from the one or more communication devices associated with the one or more first users and the one or more databases;
compare the one or more medical data comprising the one or more magnetic resonance imaging (MRI) scans of the one or more second users, with the one or more historical medical records associated with the one or more second users, using the computer vision model; and
analyze the one or more visual information associated with the magnetic resonance imaging (MRI) scans of the one or more second users, indicating the cancer symptoms, based on comparison of the one or more medical data comprising the one or more magnetic resonance imaging (MRI) scans of the one or more second users, with the one or more historical medical records associated with the one or more second users, using the computer vision model.

11. The artificial intelligence based (AI-based) cancer detection system of claim 10, further comprising a training subsystem configured to train the computer vision model based on one or more first training datasets associated with the one or more visual information of at least one of: the one or more medical data and the one or more historical medical records.

12. The artificial intelligence based (AI-based) cancer detection system of claim 8, wherein the training subsystem is further configured to train the artificial intelligence (AI) model, and wherein in training the artificial intelligence (AI) model, the training subsystem is configured to:
obtain one or more second training datasets associated with at least one of: the one or more medical data and the one or more historical medical records, wherein the one or more second training datasets comprise the one or more first training datasets being processed by the computer vision model;
assign one or more training weights to the one or more second training datasets based on priority of each data associated with at least one of: the one or more medical data and the one or more historical medical records;
fine-tune the one or more training weights based on the one or more second training datasets that indicate information associated with tumor lifecycle to determine whether a training subsystem configured with the artificial intelligence (AI) model analyzes one or more cancer patterns without bias from tumor progression stages; and
detect the type of the cancer comprising at least one of: benign tumor lifecycle and malignant tumor lifecycle based on the fine-tuned one or more training weights assigned to the one or more second training datasets.

13. The artificial intelligence based (AI-based) cancer detection system of claim 12, wherein the training subsystem is further configured to perform a reinforcement refinement process by assigning one or more tokens for each second training dataset to analyze each medical data, upon failure of detection of the malignant tumor lifecycle,
wherein in performing the reinforcement refinement process, the training subsystem is configured to retrain the artificial intelligence (AI) model with one or more corrected training weights integrating one or more feedback, to optimize an accuracy of the detection of the type of the cancer.

14. The artificial intelligence based (AI-based) cancer detection system of claim 8, wherein the training subsystem is further configured to:
obtain the one or more data comprising the one or more textual information associated with at least one of: the one or more blood test results and the one or more genetic information, of the one or more second users;
analyze at least one of: one or more terminologies, one or more concepts, and one or more contexts, associated with the type of the cancer from the one or more textual information associated with at least one of: the one or more blood test results and the one or more genetic information, of the one or more second users, using the artificial intelligence (AI) model, wherein the artificial intelligence (AI) model comprises one or more natural language processing (NLP) models; and
generate one or more insights associated with the type of the cancer from at least one of: the one or more medical data and the one or more historical medical records, based on the analysis of the at least one of: the one or more terminologies, the one or more concepts, and the one or more contexts, associated with the type of the cancer, using the artificial intelligence (AI) model.

15. A non-transitory computer-readable storage medium having instructions stored therein that when executed by one or more hardware processors, cause the one or more hardware processors to execute operations of:
obtaining one or more data from at least one of: one or more communication devices associated with one or more first users, and one or more databases, wherein the one or more data comprise at least one of: one or more medical data and one or more historical medical records, associated with one or more second users, and wherein the one or more medical data associated with the one or more second users comprise at least one of: one or more magnetic resonance imaging (MRI) scans, one or more blood test results, and one or more genetic information, of the one or more second users;
analyzing one or more visual information associated with the magnetic resonance imaging (MRI) scans of the one or more second users, indicating cancer symptoms by comparing the one or more medical data with the one or more historical medical records, using a computer vision model;
analyzing one or more textual information associated with at least one of: the one or more blood test results and the one or more genetic information, of the one or more second users, and the one or more visual information associated with the magnetic resonance imaging (MRI) scans being analyzed by the computer vision model, using an artificial intelligence (AI) model;
detecting a type of the cancer in the precancerous phases based on the analysis of the one or more visual information associated with the magnetic resonance imaging (MRI) scans of the one or more second users, and one or more textual information associated with at least one of: the one or more blood test results and the one or more genetic information, of the one or more second users, using the artificial intelligence (AI) model; and
providing an output of the type of the cancer in the precancerous phases in form of one or more reports through one or more user interfaces associated with the one or more communication devices of the one or more first users.

16. The non-transitory computer-readable storage medium of claim 15, wherein detecting, by the one or more hardware processors, the type of the cancer in the precancerous phases comprises comparing the one or more data obtained from at least one of: the one or more communication devices associated with the one or more first users and the one or more databases, with one or more trained datasets obtained from the artificial intelligence (AI) model.

17. The non-transitory computer-readable storage medium of claim 15, wherein analyzing the one or more visual information associated with the magnetic resonance imaging (MRI) scans of the one or more second users, using the computer vision model, comprises:
obtaining the one or more medical data comprising the one or more magnetic resonance imaging (MRI) scans of the one or more second users, from the one or more communication devices associated with the one or more first users and the one or more databases;
comparing the one or more medical data comprising the one or more magnetic resonance imaging (MRI) scans of the one or more second users, with the one or more historical medical records associated with the one or more second users, using the computer vision model; and
analyzing the one or more visual information associated with the magnetic resonance imaging (MRI) scans of the one or more second users, indicating the cancer symptoms, based on comparison of the one or more medical data comprising the one or more magnetic resonance imaging (MRI) scans of the one or more second users, with the one or more historical medical records associated with the one or more second users, using the computer vision model.

18. The non-transitory computer-readable storage medium of claim 15, further comprising training the artificial intelligence (AI) model, by:
obtaining one or more second training datasets associated with at least one of: the one or more medical data and the one or more historical medical records, wherein the one or more second training datasets comprise the one or more first training datasets being processed by the computer vision model;
assigning one or more training weights to the one or more second training datasets based on priority of each data associated with at least one of: the one or more medical data and the one or more historical medical records;
fine-tuning the one or more training weights based on the one or more second training datasets that indicate information associated with tumor lifecycle to determine whether a training subsystem configured with the artificial intelligence (AI) model analyzes one or more cancer patterns without bias from tumor progression stages; and
detecting the type of the cancer comprising at least one of: benign tumor lifecycle and malignant tumor lifecycle based on the fine-tuned one or more training weights assigned to the one or more second training datasets.

19. The non-transitory computer-readable storage medium of claim 18, further comprising performing a reinforcement refinement process by assigning one or more tokens for each second training dataset to analyze each medical data, upon failure of detection of the malignant tumor lifecycle,
wherein performing the reinforcement refinement process comprises retraining the artificial intelligence (AI) model with one or more corrected training weights integrating one or more feedback, to optimize an accuracy of the detection of the type of the cancer.

20. The non-transitory computer-readable storage medium of claim 15, further comprising:
obtaining the one or more data comprising the one or more textual information associated with at least one of: the one or more blood test results and the one or more genetic information, of the one or more second users;
analyzing at least one of: one or more terminologies, one or more concepts, and one or more contexts, associated with the type of the cancer from the one or more textual information associated with at least one of: the one or more blood test results and the one or more genetic information, of the one or more second users, using the artificial intelligence (AI) model, wherein the artificial intelligence (AI) model comprises one or more natural language processing (NLP) models; and
generating one or more insights associated with the type of the cancer from at least one of: the one or more medical data and the one or more historical medical records, based on the analysis of the at least one of: the one or more terminologies, the one or more concepts, and the one or more contexts, associated with the type of the cancer, using the artificial intelligence (AI) model.
